(19) United States
(12) Patent Application Publication
(10) Pub. No.: US 2019/0069846 A1
(43) Pub. Date: Mar. 7, 2019

(54) KNEE BRACE PROVIDING DYNAMIC DATA

(71) Applicant: Chris Dudziak, San Francisco, CA (US)

(72) Inventor: Chris Dudziak, San Francisco, CA (US)

(21) Appl. No.: 16/178,210

(22) Filed: Nov. 1, 2018

Related U.S. Application Data
(63) Continuation-in-part of application No. 14/440,004, filed on Apr. 30, 2015, now Pat. No. 10,105,251.
(60) Provisional application No. 61/737,659, filed on Dec. 14, 2012.

(51) Int. Cl. A61B 5/00; A61B 5/11; A61F 5/01

(52) U.S. Cl. CPC A61B 5/6812; A61B 5/1114; A61B 5/002; A61B 5/6898; A61B 2562/046; A61F 5/0111; A61F 5/0118; A61B 2562/0219; A61F 5/0109

(57) ABSTRACT

A flexible knee brace has integrated accelerometers. The knee brace is a sleeve having panels that provide support to the knee. Accelerometers may be affixed with one or a pair of the braces at different locations on the braces to obtain and evaluate data regarding movement of the knee.

### CROSS REFERENCE

This application is a continuation-in-part of U.S. patent application Ser. No. 14/440,004 entitled Flexible Support Brace, which was the U.S. National Phase of International Application No. PCT/US2013/075066, which in turn claimed priority from U.S. Provisional Patent Application No. 61/737,659, the entirety of which is incorporated herein by reference.

### TECHNICAL FIELD

This disclosure relates generally to a flexible support brace for therapeutic support and resistance to movement in joints, such as the knee, ankle, wrist and elbow.

### BACKGROUND

Joint injuries are common for both competitive and recreational athletes, or for those suffering from arthritis. For example, a sprain is a stretching or tearing of a ligament that joins one bone to another, and may be caused by a fall, twist or blow to the joint, while a strain is a twist, pull or tear of a muscle or tendon (tendons connect muscle to bone) caused by stretching or contracting the muscle or tendon more than normal. Other types of injuries, such as bursitis, tendonitis, or repetitive injuries (carpal tunnel syndrome), may be mild or severe.

While the knee is probably the most commonly injured joint, the ankle, wrist and elbow are also frequently injured. Taking steps to prevent injury is important, but once a joint injury has occurred, keeping the joint stable is the primary goal for rehabilitation. To that end, there are a number of commercial products that seek to provide support. For example, the Ace® bandage is a well-known elastic wrap that is used to wrap around an injured joint, providing some degree of uniform support throughout the injured area. However, such a bandage does not provide focused support and/or resistance to movement based on the nature of the injury or the particular joint movement.

There are also elastic braces sold by Ace and others specifically designed for the ankle, knee, elbow or wrist, for example. However, these location-specific braces are uniform in material construction, and still do not provide adequate focused support and/or resistance to joint movement based on the nature of the injury or the particular joint movement.

Thus, it would thus be desirable to have an improved brace that is focused on providing support and/or resistance to specific joint movements.

### BRIEF DESCRIPTION OF THE DRAWINGS

FIG. 1 is a schematic diagram of a human anatomical subject illustrating the three primary planes of joint movement.

FIG. 2A is a front perspective view of a generic brace structure.

FIG. 2B is a top plan view of an alternative brace structure having one side formed in a trapezoidal shape.

FIG. 3A is a front perspective view of a knee brace.

FIG. 3B is a front plan view illustrating a knee joint in four different rotational positions.

FIG. 3C is a front perspective view of the knee brace of FIG. 3A showing the axis of knee joint rotation.

FIG. 3D is a front plan view of a human right leg showing the myofascial meridians running through the leg.

FIG. 3E is a front plan view illustrating the knee brace of FIG. 3A over the knee joint of FIG. 3B in one of the bent positions.

FIG. 4 is a front perspective view of an ankle brace.

FIG. 5 is a front perspective view of a wrist brace.

FIG. 6 is a rear perspective view of an elbow brace.

FIG. 7 is a schematic diagram illustrating knee braces with integrated accelerometers on a human subject.

FIG. 8A is a front perspective view of a knee brace having integrated accelerometers.

FIG. 8B is an exploded detail view of a portion the knee brace shown in FIG. 8.

FIG. 9A is a front perspective view of a knee brace having a fabric strap threaded through holes in the knee brace and an accelerometer contained within the fabric strap.

FIG. 9B is a side plan view of an alternative wrap-around strap having a cavity to contain the accelerometer.

FIG. 10 is a flow chart illustrating a process for collecting and evaluating accelerometer data.

FIGS. 11A, 11B and 11C are tables presenting sample predicted and assessed acceleration data for knee-related movements of the user in the x, y and z directions, respectively.

FIGS. 12A and 12D are graphs plotting the predicted and assessed acceleration data from FIG. 11A for knee-related movements of the user in the x direction, respectively.

FIGS. 12B and 12E are graphs plotting the predicted and assessed acceleration data from FIG. 11B for knee-related movements of the user in the y direction, respectively.

FIGS. 12C and 12F are graphs plotting the predicted and assessed acceleration data from FIG. 11C for knee-related movements of the user in the z direction, respectively.

### DETAILED DESCRIPTION

### 1. Overview

This disclosure describes a flexible brace for stabilizing and supporting an underlying joint. The brace is formed from a flexible elastic material such as a silicone or polyurethane. The brace has a generally annular structure with a main section formed with a pattern of large holes disposed throughout the main section, and at least one support section formed with a pattern of small holes aligned along a plane of motion of the underlying joint or a meridian proximate to the joint. The smaller holes provide an increased volume of material that supports and stabilizes the underlying joint.

### 2. Joints and Myofascial Meridians

The human body may be considered an ordered collection of many bones, some connected by joints that permit bodily movement, such as knee, ankle, elbow and wrist, which are the initial focus of embodiments of the braces described herein. Of course, there are connective tissues such as ligaments, synovial fluid, etc., that facilitate joint operation. The concept of myofascial meridians is used to describe lines connective tissue that run throughout the body, linking all parts of the body, and providing the organized structural forces required for motion. (See, e.g., Myers, T., “Anatomy Trains,” Journal of Bodywork and Movement Therapies, vol. 1, issue 2, pp. 91-101, January 1997). All of the foregoing can be taken into account, as further discussed below, in constructing a suitable brace to provide support for different physical issues of the user.

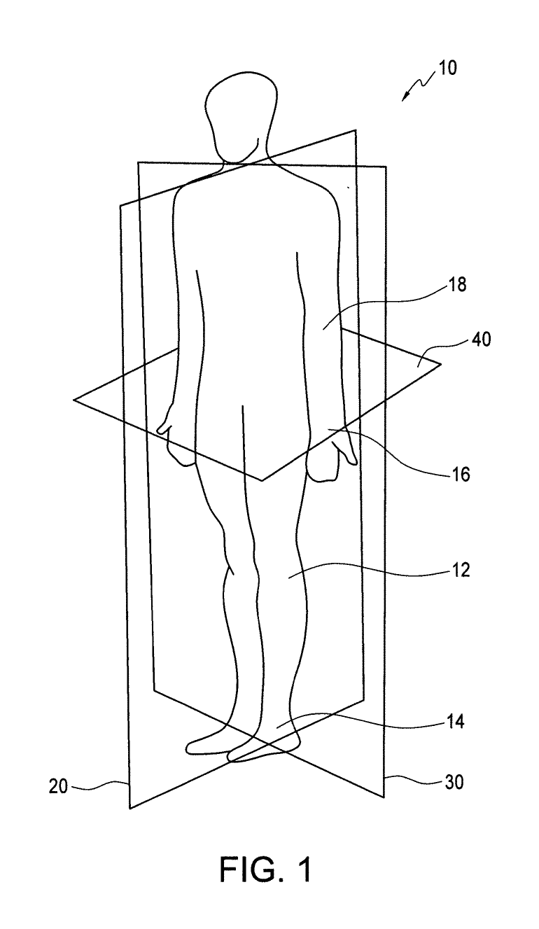

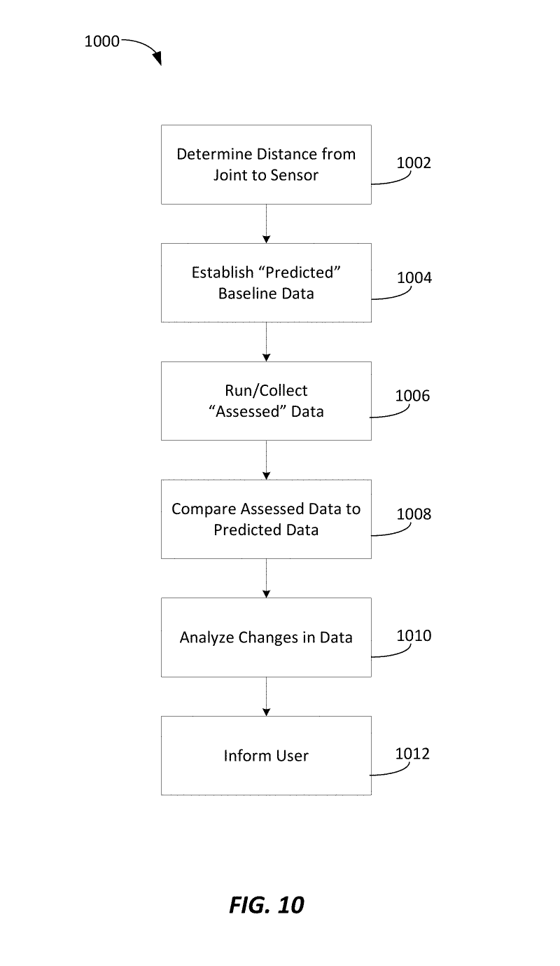

It is helpful to provide a frame of reference for physical descriptions, and thus FIG. 1 illustrates an anatomical FIG. 10 having a knee 12, ankle 14, wrist 16 and elbow 18. The three primary planes of movement can be described as: the sagittal plane 20, a vertical plane that divides the body into left half and right half; the frontal plane 22, a vertical plane perpendicular to the sagittal plane that divides the body into an anterior or ventral (front) half and a posterior or dorsal (rear) half; and the transverse plane 24, a horizontal plane that divides the body into upper and lower portions.

The most common joint movement is flexion and extension in the sagittal plane, typified by the hinge joint of the elbow, the modified hinge joint of the knee, and the condyloid joint of the wrist. The movement of the ankle hinge joint is a little more complex, including dorsiflexion (movement is the frontal plane); plantar flexion (movement in the sagittal plane); and a slight circumduction (movement in the transverse plane). Generally, the extensor muscles that create/assist the extension movement are weak compared to those that create/assist the flexion movement.

### 3. Building a Support Brace, Generally

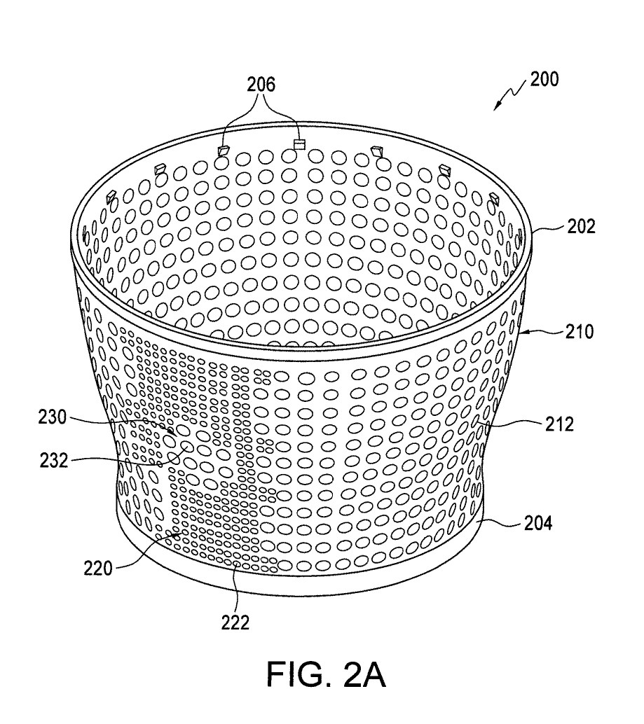

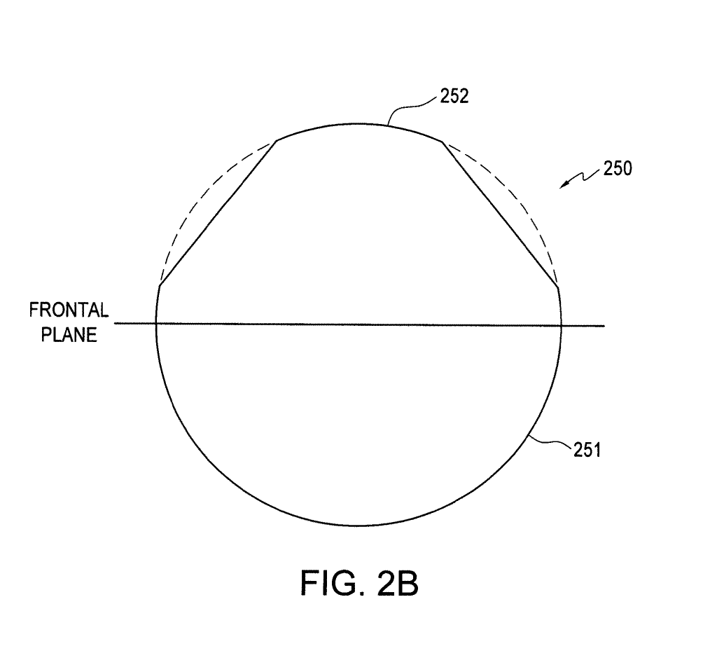

FIG. 2A illustrates one embodiment of an annular structure 200 formed as a flexible brace. The brace 200 can be formed by injection molding, for example, using a silicone material such as Mold Star® silicone rubber or other suitable elastic materials. Although brace 200 is shown as cylindrical in shape, other embodiments can be made to better fit the knee, ankle, leg, wrist, elbow, and arm, as discussed below. For example, as shown in FIG. 2B, another brace 250 could be formed with anterior half 251 formed to have a semi-circular profile and the posterior half 252 formed with a trapezoidal profile to provide a better fit over the knee, elbow, etc. Further, any such braces could be made and sold in standard sizes, such as small, medium or large, or custom made to order. The advent of 3D printing to quickly and inexpensively form custom molds may facilitate production of custom braces.

Referring back to FIG. 2A, the annular brace 200 has a top ring 202 and a bottom ring 204, both formed as solid ribbons of material around the top and bottom of the structure, with one or more sections or side panels having different size holes, such as panels 210 and 220, formed between the top and bottom rings. The side panels may be uniform in material thickness and density, but preferably, the material will vary in thickness and/or density as a means to define support portions of the brace as discussed below. For example, the panels may be molded generally to a thickness of 2.5 mm, but additional material could be added to specific support portions. For example, panel 210 covers most of the area between the rings 202, 204, and may be molded with a standard thickness of 2.5 mm, while smaller panel 220 may be molded with an increased thickness of up to 5 mm to enhance the ability of the smaller panel 220 to both stabilize the underlying joint and to store energy for resistance to movement of the joint. Friction bumps 206 may be formed on the inside portion of the top ring 202 to help grip the body above the joint and keep the brace from slipping.

In this embodiment, side panel 210 has a pattern of large holes 212 formed throughout the panel. For example, the large holes 212 may be formed to have a diameter of 12.7 mm (½ inch). Side panel 220 covers a smaller, specifically targeted area of the brace, e.g., a vertical section between the rings 202, 204, and has a pattern of smaller holes 222 formed throughout that section, for example, with a diameter of 6.35 mm (¼ inch). By making the holes 222 smaller, the side panel 220 or support panel has more material disposed through that section than side panel 210 and can therefore provide more support through a range of motion of the underlying joint. Thus, the support panel 220 should be formed along a line or section of the brace that is coplanar with the plane of motion for the underlying joint, on the anterior side of the joint. A circular section 230 having large holes 232 may be formed in the middle of the smaller hole section 220 as less restrictive area for the knee cap (patella) or the elbow, for example. More than one support panel may be formed in a brace to provide support along multiple planes of motion. Further, as noted above, an increased thickness of material may be used in regions having support panels. Alternatively, or in addition, support panels may be formed along one or more meridians of the body.

Thus, in general, a flexible brace can be designed to control the range of motion for any joint. The use of thinner and thicker portions of material in the brace, combined with the use of larger and smaller holes, can be engineered for particular physical issues to provide appropriate joint stabilization as well as energy storage to resist undesirable joint movements.

Although commercial processes are likely to create and use standard injection type molds, the emergence of 3D printing processes may allow a variety of molds to be easily and inexpensive built with amazing accuracy, in the shape of anything from a straight cylinder to a bent elbow. Software to create 3D objects is readily available, such as Adobe® Photoshop CS6 Extended software with 3D modeling option. Further, 3D printers are also readily available, such as the MakerBot Replicator 2 3D printer, or the FlashForge 3D printer. Such customization will enable the production of braces to control/stabilize any range of motion for any limbs/joints. Further, although 3D printing is still in its infancy, it is conceivable that it could be used to produce the actual braces rather than just the molds.

Creating an effective brace involves two steps. First, two fixed points are selected on the limbs to which the brace will be secured, then, material is formed between the two points so as to create a smooth surface against the skin. The volume of the material may be varied in different planes of movement. Any material in linear series will be fixed at the two points on the limb and stretched over the instantaneous axis of rotation for the joint, thus decelerating the effective moment arm that acts around the axis of rotation.

Because the two points are fixed, the flexible material will lengthen away from the joint center as the joint moves through a range of motion that changes the joint angle. At the joint rest or starting position, no tension is stored in the brace. However, at the end position, elastic energy will be stored in the brace.

The Poisson effect is an important mechanical characteristic that relates to the forces that are applied and created across a cross-section of material. Basically, when a body is subject to a uniaxial stress in one planar direction, a strain is created in the other two perpendicular planes that increases the dimension of the material in those perpendicular planes. The converse is also true. For example, a body experiencing a tensile load which generates an increase in its axial dimensions also generates a decrease in its transverse dimensions. Thus, by having top and bottom rings secured at a fixed point relative to the joint, the brace will self-tighten onto the limb thereby helping to stabilize the underlying joint(s) and hold the brace in place on the surface of the skin, in combination with the use of friction bumps on the inside of the rings.

As the brace de-forms about the joint center, the moment arm of the joint is pushed out to the surface of the skin, thereby increasing the load applied to the brace. The change in the joint angle is proportional to the amount of tension stored in the brace, and as the joint flexes, more energy is stored in the brace. Further, due to the Poisson effect, the tension is passed laterally through the brace wall as well as circumferentially around the brace.

In general, any material that exists anterior to the joint center will decrease knee flexion, and any material located posterior to the joint center will decrease knee extension. Likewise, material located laterally to the knee will decrease varus loading, while material located medially to the knee will decrease valgus loading.

If the brace has a uniform consistency and thickness, the wall created against the skin makes it difficult to differentiate the volume of material and to vary loads in specific directions. However, by using holes in the material and varying the circumference of the holes, effective stabilization and support for the underlying joint can be created. Thus, the use of larger holes presents less elastic material in series thereby creating less resistance. However, the use of smaller holes puts more elastic material in series thereby creating more resistance in a given direction of movement. The ability to create a linear resistance in a specified direction applies to all three planes of movement and is essential to creating smooth and efficient movement patterns.

Increasing the volume of material in selected areas between the rings enables coordinating pressure over joint centers as they move through ranges of motion. Advantageously, the volume of material can be increased by forming “straps” of additional material on the surface of the brace in the direction the myofascial meridians. The straps are formed as part of the initial molding of the brace. As the joint goes through flexion and extension, tension is passed though the elastic matrix pulling on the straps to secure them as well as lengthening them across the instantaneous joint center, much like bending a beam.

The placing of more material in line with the myofascial meridians helps to secure the brace in place as well as help support and control the dynamic nature of the joint center and direct force over or in a plane of motion.

### 4. Support Brace for Knee

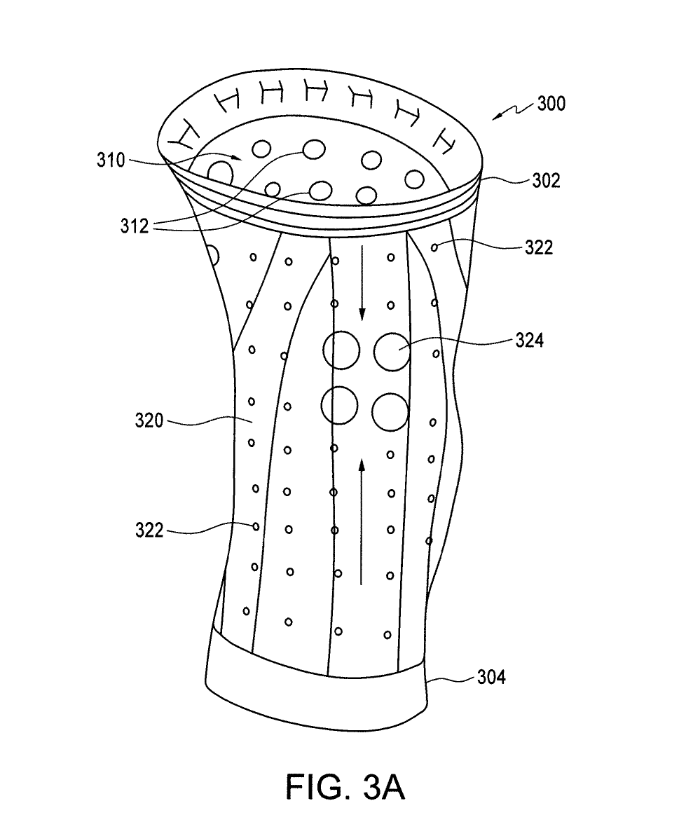

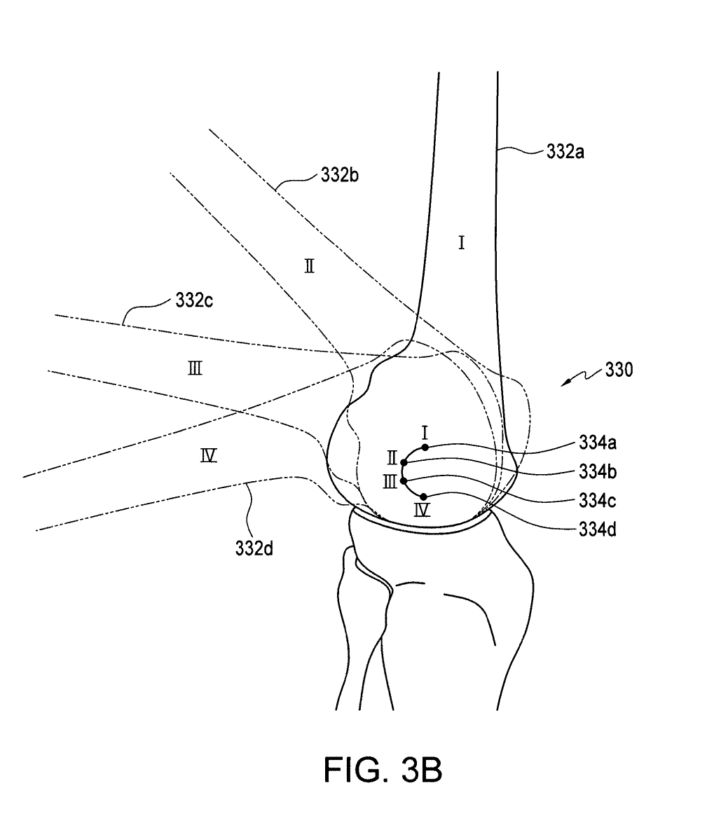

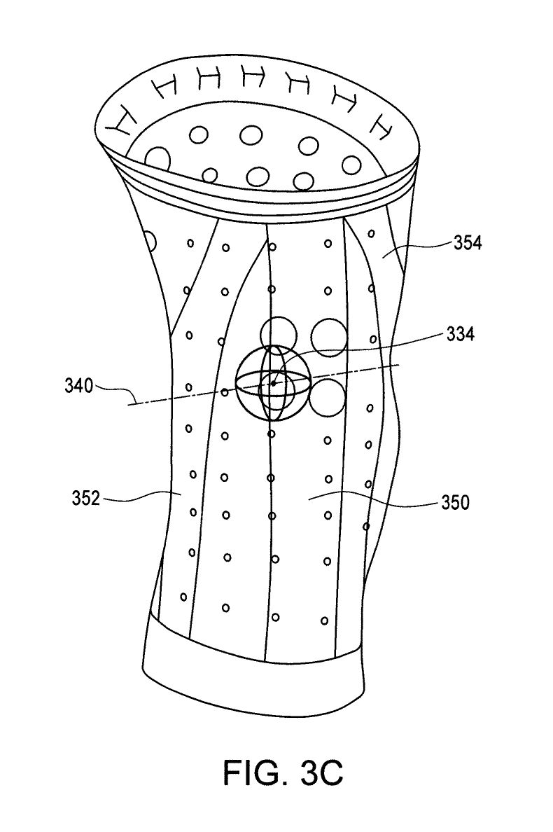

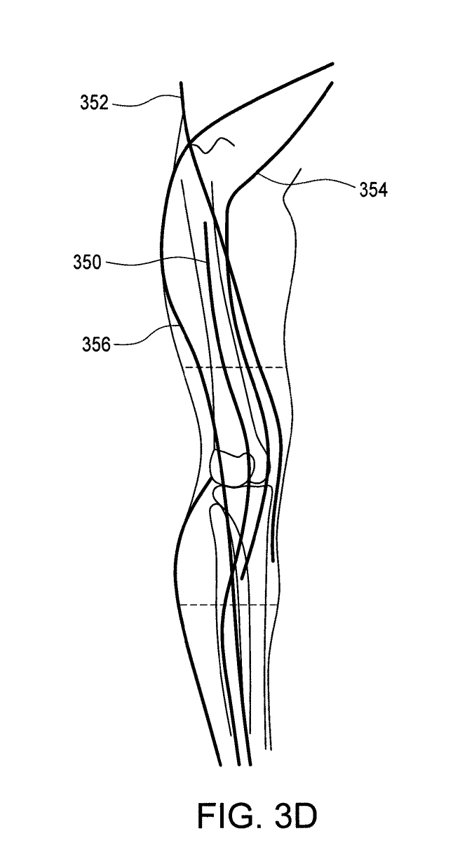

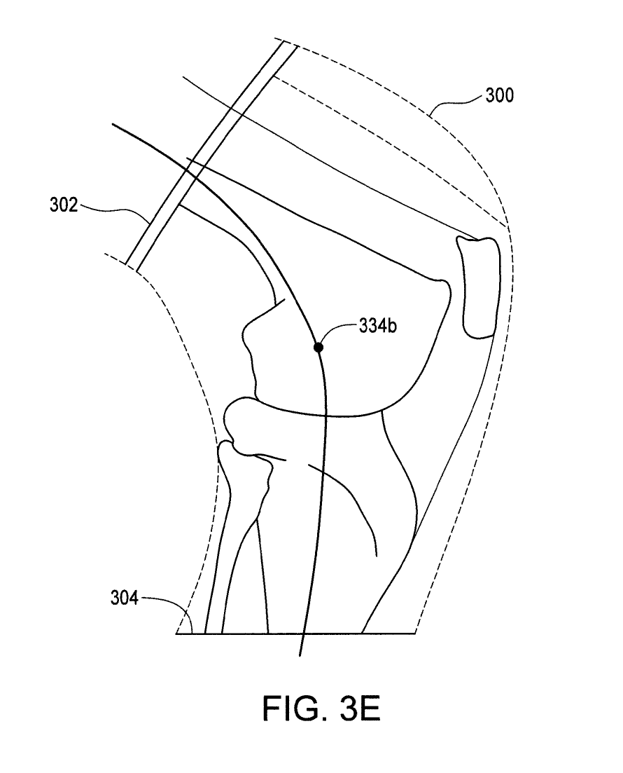

FIG. 3A illustrates a brace 300 formed to better fit and support the knee. For example, the top ring 302 and corresponding top portion of the brace may have a larger diameter to better fit above the knee, and a slight rearward tilt. The bottom ring 304 and corresponding bottom portion of the brace have a smaller diameter to better fit below the knee. The posterior portion 310 of the brace 300 has a pattern of large diameter holes 312, while the anterior portion 320 of the brace has a pattern of smaller diameter holes 322. Further, several larger holes 324 are formed in correspondence with the patella. Thus, the smaller holes 322 provide a linear series resistance on the anterior side to flexion and extension movements of the knee joint.

FIG. 3B illustrates the knee joint 330 with the femur 332 in four different positions. In position I (332a), the leg is straight with the knee in full extension. In position IV (332d), the leg is bent at the knee in full flexion. Position II (332b) and position III (332c) are intermediate positions. When building any brace, the center of rotation for the underlying joint is a key location. In some joints, however, like the knee, the center of joint rotation is not fixed in one spot, but moves with a flexion or extension movement. This movement is also illustrated in FIG. 3B, where point 334a is the instantaneous center of rotation when the knee is in position I; point 334b is the instantaneous center of rotation when the knee is in position II; point 334c is the instantaneous center of rotation when the knee is in position III; and point 334d is the instantaneous center of rotation when the knee is in position IV.

FIG. 3C is similar to FIG. 3A, but includes an axis of rotation 340 around the instantaneous center point 334 superimposed on the knee brace 300. Further, the relationship of significant meridian lines to the knee joint is also shown on FIG. 3C. For example, the superficial front line (SFL) 350 is behind the center of the brace on the anterior side; the lateral line (LL) 352 is behind the outside portion of the knee joint; and the deep front line (DFL) 354 is behind the inside portion of the knee joint. These meridians are also illustrated relative to the right knee in FIG. 3D.

FIG. 3E shows the brace 300 covering the knee joint 330 in position II having center of rotation 334b, with top ring 302 snugly fit above the knee and bottom ring 304 snugly fit below the knee.

An evaluation of the performance of the knee brace was performed using a seated knee extension machine. A vertical stack of weights was loaded onto the machine, and the subject performed weighted leg extensions according to the standard control and test battery used by the National Strength and Conditioning Association (NSCA). For example, the tests started with high weight and low repetitions then moved to low weight and high repetitions.

Four different attributes were tested, namely, anaerobic power, anaerobic endurance, aerobic strength, and aerobic endurance. The trials included a 5-minute warm-up on an exercise bicycle with no brace; then a control battery with no brace; and finally, a test battery with brace. The trials indicated that, while wearing the knee brace, an increase of approximately 35% in anaerobic power was observed; an increase of approximately 37% in anaerobic endurance was observed; an increase of approximately 38.5% in aerobic strength was observed; and an increase of approximately 25% in aerobic endurance was observed;

### 5. Support Brace for Ankle

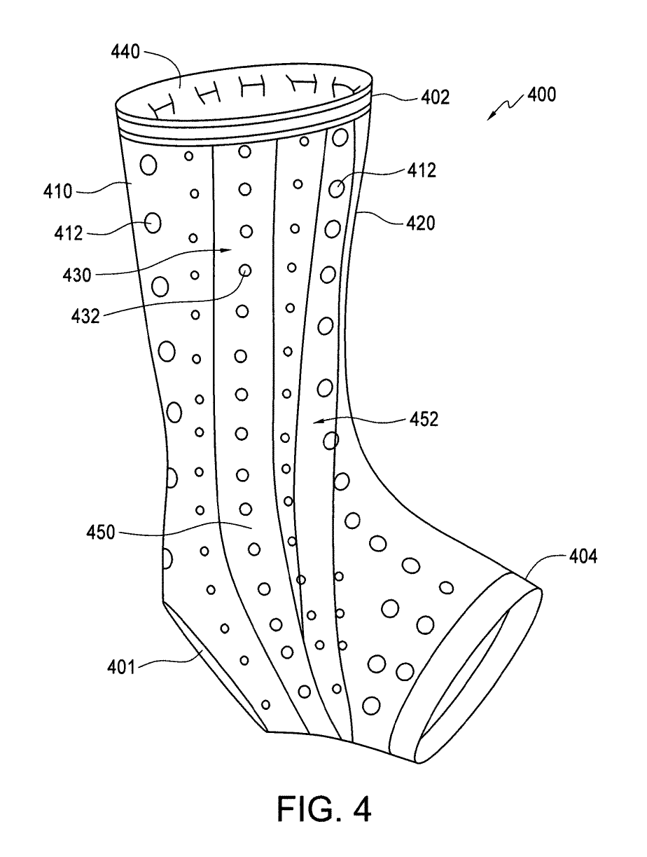

FIG. 4 illustrates a brace 400 formed to better fit and support the ankle, the brace is formed in a L-shape between the top ring 402 and the bottom ring 404 to match the shape of the foot, with a heel opening 401. The top ring 402 is sized to fit snugly above the ankle, and the bottom ring 404 is sized to fit around the foot. The posterior portion 410 and the anterior portion 420 of the brace 400 have patterns of large diameter holes 412, while the inside portion 430 and outside portions 440 have patterns of smaller diameter holes 432. The smaller holes 432 are vertically oriented to follow the spiral line lateral meridians 450 on both the medial and lateral portions of the ankle, while the larger holes 412 are vertically oriented on the posterior side of the ankle and also vertically oriented to follow the superficial front line meridian 452 on the anterior side. The smaller holes 432 provide a linear series resistance on both the medial and lateral portions of the ankle to rotational movements of the ankle joint.

### 6. Support Brace for Wrist

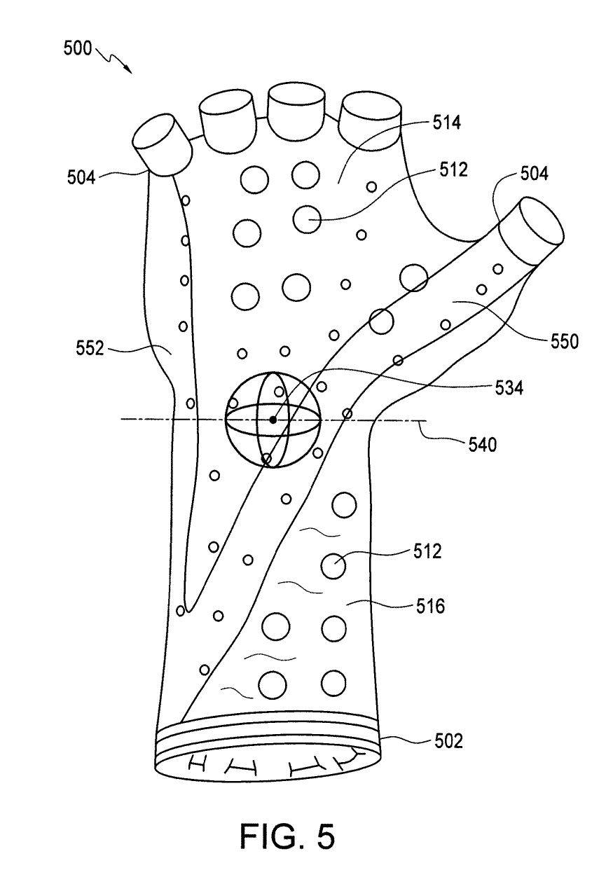

FIG. 5 illustrates a brace 500 formed to better fit and support the wrist. The brace 500 is formed like a glove, with five ringed finger openings 504 at one end and a top ring 502 sized to fit snugly above the wrist. Patterns of large diameter holes 512 are formed through the palm area 514 and through the area 516 below the thumb. However, it may be desirable to have an open area of no material is the palm area to enable gripping a racket or club, or an area of less material for an injury such as carpal tunnel, which does not require restrictive pressure from the palm side. Area 516 below the thumb is a compressed zone because the hand pronates around axis 540 that runs through the joint center 534, and supinates in the resting position. Patterns of smaller holes 514 are oriented to follow the deep front arm line meridian 550 that runs from the outside of the wrist across to the end of thumb, and also to follow the deep back arm line 552 that runs along the outside of the hand. The smaller holes 514 provide a linear series resistance on both the inside and outside of the wrist to rotational movements of the wrist joint.

### 7. Support Brace for Elbow

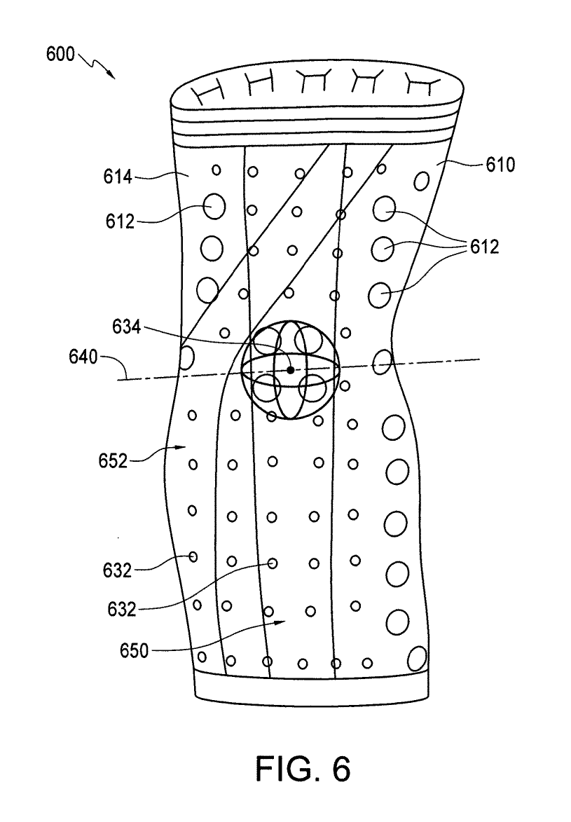

FIG. 6 illustrates a brace 600 formed to better fit and support the elbow. The brace 600 is formed much like the knee brace 300, with a top ring 602 sized to fit snugly above the elbow and a bottom ring 604 sized to fit snugly below the elbow. Patterns of large diameter holes 612 are formed vertically along the entire length of the anterior section 610 and also in the posterior section 614 above the elbow. Patterns of smaller holes 632 are oriented to follow the superficial back arm line meridian 650 that runs straight through the elbow, and also to follow the deep back arm line meridian 652 that runs from the outside of the elbow on the anterior side across to the inside of the elbow on the posterior side. The smaller holes 632 provide a linear series resistance to rotational movements of the elbow joint.

### 8. Dynamic Data

In addition to providing effective support for underlying joints, any of the braces described herein can be fitted with one or more digital sensors to monitor the activity and/or movement of the brace, in particular, to describe movement of the underlying joint. Further, the sensors may share data with other applications and other digital devices, such as the user's smartphone, tablet, wrist-worn device (e.g., FitBit, AppleWatch), desktop computer, etc., using standard communication protocols (e.g., Bluetooth).

For example, one or more accelerometers may be attached to a brace in order to dynamically generate data regarding movement of the relevant area when the brace is in use, such as when the user wears a brace on one or both knees while jogging. The data can be received by a user software application and plotted in a chart and/or on a three-dimensional graph in order to illustrate the movement of the joint, in this case the knee, relative to the sagittal plane (x-axis), the frontal plane (y-axis) and the transverse plane (z-axis). The data may then be analyzed in order to determine relevant characteristics of the user's running patterns, and in particular, is the user experiencing fatigue or weakness in the joint that can be detected and addressed.

FIG. 7 is a simplistic depiction of a pair of knee braces 700, 701 worn on a user's knees and made from flexible elastic material as described above. Each knee brace has an upper ring 702 sized to fit snugly above the knee and a lower ring 704 sized to fit snugly below the knee. Further, a plurality of panels or sections are affixed between the upper ring 702 and the lower ring 704, including at least a posterior panel 710 behind the knee, an anterior panel 712 in front of the knee, a lateral panel 714 on the outside of the knee, and a medial panel 716 on the inside of the knee. As described above, the anterior panel 712 has an increased volume of material relative to the posterior panel 714, which may be implemented by forming smaller holes on the anterior panel and larger holes on the posterior panel. However, the panels may be formed and/or patterned in numerous different ways to provide appropriate support as needed for a particular user.

In one embodiment, a first pair of accelerometers 720, 721 is affixed on a lateral panel 714 of the left knee brace 700. The first accelerometer 720 is affixed in a proximal location near the upper ring 702 and the second accelerometer 721 is affixed in a distal location near the lower ring 704. In another embodiment, a second pair of accelerometers may be affixed on a medial panel in correspondence with the first pair of accelerometer positions on the same knee brace. For example, right knee brace 701 is shown with accelerometers 722, 723 disposed on the medial panel 716, with the third accelerometer 722 affixed in a proximal location near the upper ring 702 and the fourth accelerometer 723 affixed in a distal location near the lower ring 704.

By comparing the acceleration at the proximal location with the acceleration at the distal location, the user's gait can be analyzed and insight gained to provide recommendations made on how to improve the gait or adjust for changes in the gait.

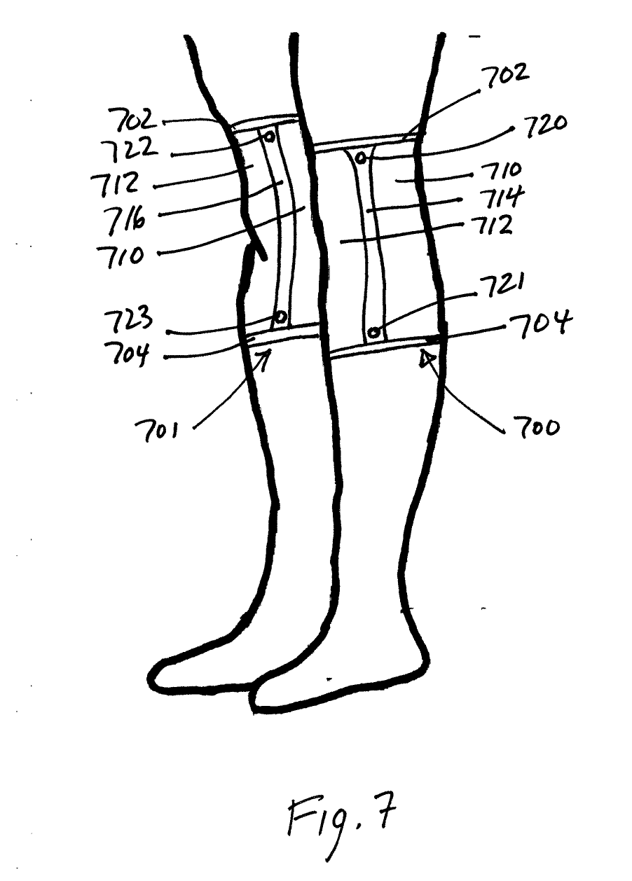

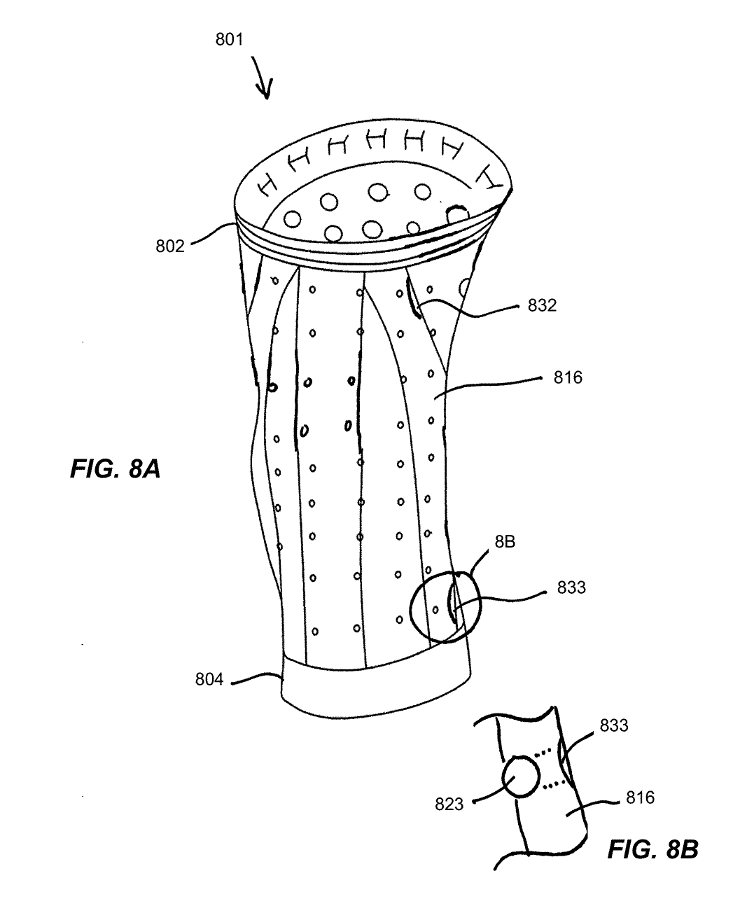

FIG. 8A illustrates a more detailed example of a right knee brace 801 as modified to include a pair of accelerometers coupled with the brace. As noted above, knee brace 801 includes an upper ring 802 and a lower ring 804, with panels affixed between the upper and lower rings including medial (inside) panel 816. In one embodiment, a first slot 832 is formed in a proximal location at the seam of the medial panel 816 near the upper ring 802 to receive and store, for example, the third accelerometer 722, and a second slot 833 is formed in a distal location at the seam of the medial panel near the lower ring 804 to receive and store, for example, the fourth accelerometer 723. The corresponding lateral (outside) panel has a similar construction.

The slots 832, 833 may be formed as a pocket with a thin opening when molding the brace such that the accelerometers 722, 723, respectively, can be slipped into the corresponding slot and securely retained, as shown in FIG. 8B, or removed in order to replace it or to replace the battery.

One of the advantages of making the braces with the pockets in fixed sites on the brace is that the distance from the kneecap center of the brace to each accelerometer site is known, and knowledge of this distance is required to properly calculate accelerations at the respective sites.

Other methods could be used to couple the accelerometers with the brace. For example, if the braces are made without embedded accelerometers, the user can simply attach accelerometers at appropriate locations, such as the proximal and distal locations described above, using a strap and/or fasteners of some kind to secure the device(s) to the brace.

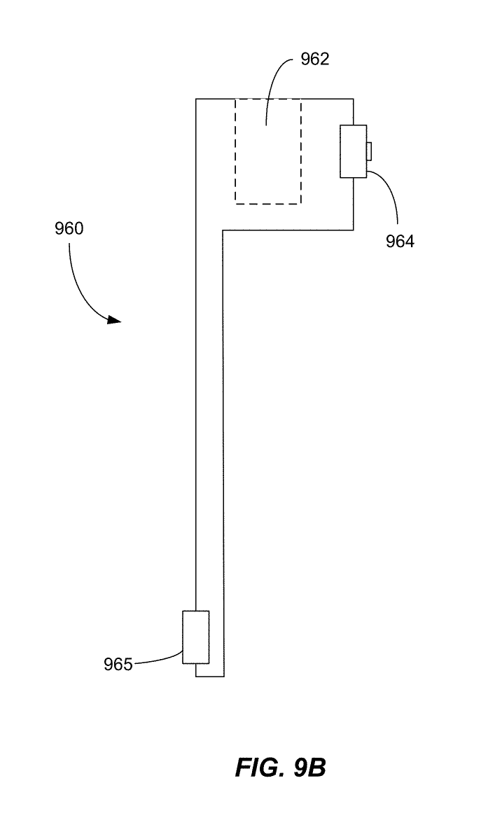

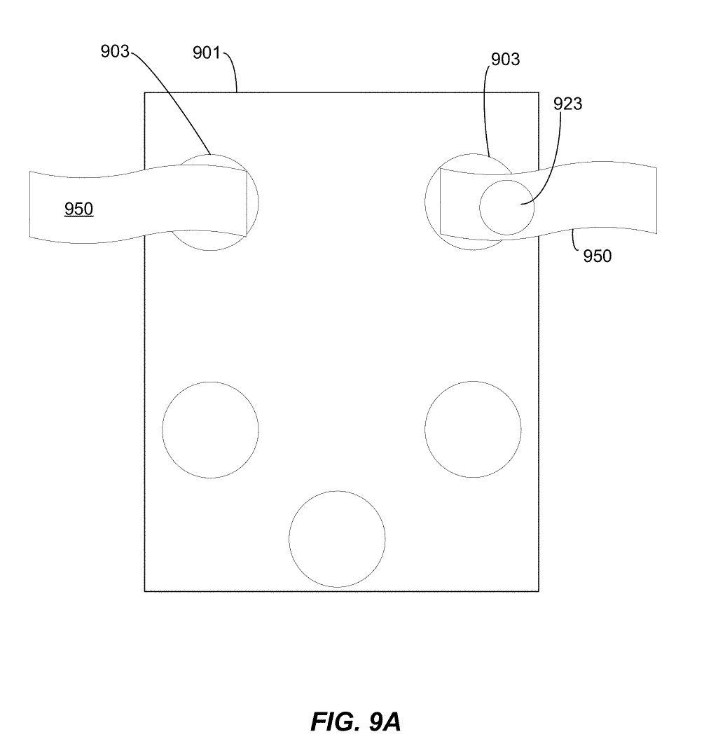

In one embodiment, illustrated in FIG. 9A, a narrow length of loose fabric 950 could be threaded through a spaced-apart pair of the holes 903 in brace 901 and simply pulled tight or tied off to itself, with an accelerometer 923 contained within the fabric. Alternatively, the strap may be fitted with a simple closure mechanism, such as male/female button closure, hook and loop fastener, etc.

In another embodiment, illustrated in FIG. 9B, a heavy-duty strap 960 is formed with a cavity or pouch 962 at one end for securely holding an accelerometer. The strap 960 has a length that is adequate for wrapping around the brace, such 8 cm for a knee brace. A pair of mating closure mechanisms 964, 965 are affixed at respective ends of the strap 960. The closure mechanisms 964, 965 may be any type of connector, closure or fastener, such as buttons, or hook and loop type fasteners.

One problem with having the user manually attach one or more accelerometers to the brace is that the distance from the kneecap center to each accelerometer site is not known with specificity. Therefore, in this situation, the relevant distance must be determined. One way to determine the distance is to place a standard size reference device adjacent the brace, take a picture of the brace (with the accelerometers being indicated on the brace in some manner) and reference device, and upload the image to the user application. For example, a credit card has a standard size and would be a good reference device for this purpose in order to provide a measurement scale. A ruler with highly visible markings would also work. The user application will digitize the image and measurement points, then determine the relevant distances based on the referenced measurement scale. This functionality may be built into newer smartphones as a measurement tool.

Of course, it would also be possible to use strap 960 as a stand-alone device attached directly to the limb of interest rather than wrapped around a brace. However, the relevant distance from the center of the underlying joint to the accelerometer must still be determined as described above.

One example of an accelerometer is the model EMBC02 Bluetooth Low-Energy Proximity Beacon with Accelerometer, made and distributed by EM Microelectronic-Marin SA. This device is a small weatherproof disk-type enclosure measuring 30 mm in diameter by 10 mm thick, with a weight of 7 grams. The device is powered by a replaceable CR2032 3V Li coin-cell type battery.

The EMBC02 device has a “moving mode” in which a data beacon with the obtained accelerometer data can be transmitted at 100 ms intervals with a 30 m range when movement is detected until one minute after the device is still. In general, the data beacon includes data packets generated by the sensor that are 37 bits long, with 12-bit fixed-point acceleration data including 6 fractional bits in two's complement format. However, the rate sampling and transmission of the accelerometer data can be adjusted as desired, and the device also has the ability to aggregate and send data according to different configurations. The complete set of features and specifications for the EMBC02 accelerometer can be find at this link: <http://www.emmicroelectronic.com/products/wireless-rf/beacons/embc02>.

For example, the analysis may show fatigue on a consistent running surface, show height changes in the run, help analyze abnormal running patterns, etc. There are “natural” gaits for human movement that are generally well-known, such as walk, skip, jog, run and sprint, that can be used as reference points. In particular, the natural gaits define how the leg muscles are used during the gait cycle, and each of the natural gaits has a different gait pattern. (See Wikipedia's article on the human gait, at link: https://en.wikipedia.org/wiki/Gait_(human)). This information is useful in analyzing the accelerometer data.

Of course, a typical therapy regimen is developed in coordination with the user's support group (medical, therapy, etc.) and is targeted for the user's injury recovery. To that end, the user's rehabilitation plan is usually fairly specific in describing the types of exercises and movements that the user should perform and the performance expectations.

Particularly in the later stages of rehabilitation from an injury, an athlete may feel no pain, but the performance is still not what the user desires or expects. Therefore, if the athlete pushes too hard too soon, further injury is possible. Thus, when the connective tissues have not had adequate time to heal, the use of the knee brace providing accelerometer data can help the user identify specifically when and how they are getting fatigued during a run. This information helps to better inform the rehabilitation process for all concerned in injury treatment: the user, therapist, doctor, etc.

Once data is collected from the accelerometer, it may be processed, coordinated and evaluated in order to analyze the gait of the user. Once the user's gait motion is understood, recommendations can be provided to the user with regard to improvements or adjustments in running technique, for example. In addition, understanding the gate motion of the user can provide information that may help to create a better, more supportive knee brace for the user. That is, the location of the support panels having smaller holes (more material) can be customized for the user to emphasize support where needed for this particular user.

In one embodiment, the user carries a smartphone, for example, wearing it at the hip. Most modern smartphones include an accelerometer, and thus, the user can receive and collect the data from the knee brace accelerometers into the smartphone in order to coordinate and analyze the data from any and all of the accelerometers. It is certainly contemplated that additional accelerometers may be incorporated into the ankle brace, the elbow brace, the wrist brace, or in other attachments (cap/helmet), as a means to establish a much more extensive profile of the user's physical response during any type of exercise. Locating the smartphone at the user's hip while working out is useful to provide more data regarding the user's movement patterns, in particular, adding the hip motion to the knee motion. Further, the EMBC02 accelerometer has a compatible smartphone application that may be downloaded for an Apple device or an Android device and used to integrate and coordinate data received from the accelerometers. A software routine may be used to plot and analyze the accelerometer data in accord with programmed instructions, or in coordination with human evaluation.

FIG. 10 illustrates a process 1000 that may be implemented to utilize and analyze data from multiple accelerometers integrated with a knee brace in order to monitor performance of a user that is walking or jogging, for example. The following example is based on a walking scenario.

In step 1002, the distance from the joint center (approximated as centered under the kneecap) to each accelerometer in this user's knee brace is determined. As noted above, these distances are known in a brace that is initially formed to have embedded accelerometers in fixed positions, but are not known where accelerometers may be attached and removed by the user, and therefore must be determined.

In step 1004, knowledge regarding the current state of the user's performance while walking is required to establish a baseline for analysis. As one example, to establish a baseline for the user, the user walks one mile while wearing a knee brace with accelerometers attached at the top (proximal) and bottom (distal) portions of the brace, respectively, that is, one accelerometer is located above and one accelerometer is located below the kneecap. The data collected over the one mile effort provides a baseline and can be used as “predicted” data for the user, while the next walk by the user generates “assessed” data that will be compared to the predicted data in order to evaluate the user's performance over a longer distance, for example. The baseline data can be updated over time by integrating different walking events for the user, or different baselines may be established during the course of a user's rehabilitation.

In step 1006, the user walks again and data from the accelerometers is collected. In step 1008, the data from the new walk, i.e., the assessed data, is compared with the predicted data from the baseline walk. In step 1010, any changes from the predicted data to the assessed data are reviewed and analyzed to evaluate possible causes of the change. Finally, in step 1012, the user is informed regarding the change(s) and provided with suggestions on how to adjust or compensate or at least recognize the source for the changes.

The data collection activity is correlated with the relevant gait. Thus, for an evaluation of a walking user, the walking event consists of a series of strides having repetitive gait motions for each foot, namely: (i) heel strike, (ii) mid-foot plant, (iii) toe off, (iv) swing phase, (v) repeat. At heel strike, the heel is stationary while the upper body is moving forward; the knee is straightening out but really not accelerating. Therefore, predicted acceleration will initially be zero in all three directions. At mid-foot, the body comes to balance over that foot, and the back foot is coming up in the air and starting to swing forward. The x and y accelerations are still zero in this phase, but there is now movement in the z direction with knee moving inward and outward, or left and right along the frontal plane. At toe off, the knee is still moving in and out. During the swing phase, the leg is moving forward and the knee upward.

Data can be collected at timely intervals, e.g., one data collection every one-tenth of a second, and then divided into the different gait motions. Further, the data is collected in three relevant dimensions, namely, the forward movement or X-direction; the knee up/down or Y-direction; and the inward/outward movement of the knee on landing or Z-direction. Thus, for a one mile walk to establish a baseline, thousands of data points will be collected at regular time intervals. For a subsequent walk, likely much more than one mile, many more data points will be collected.

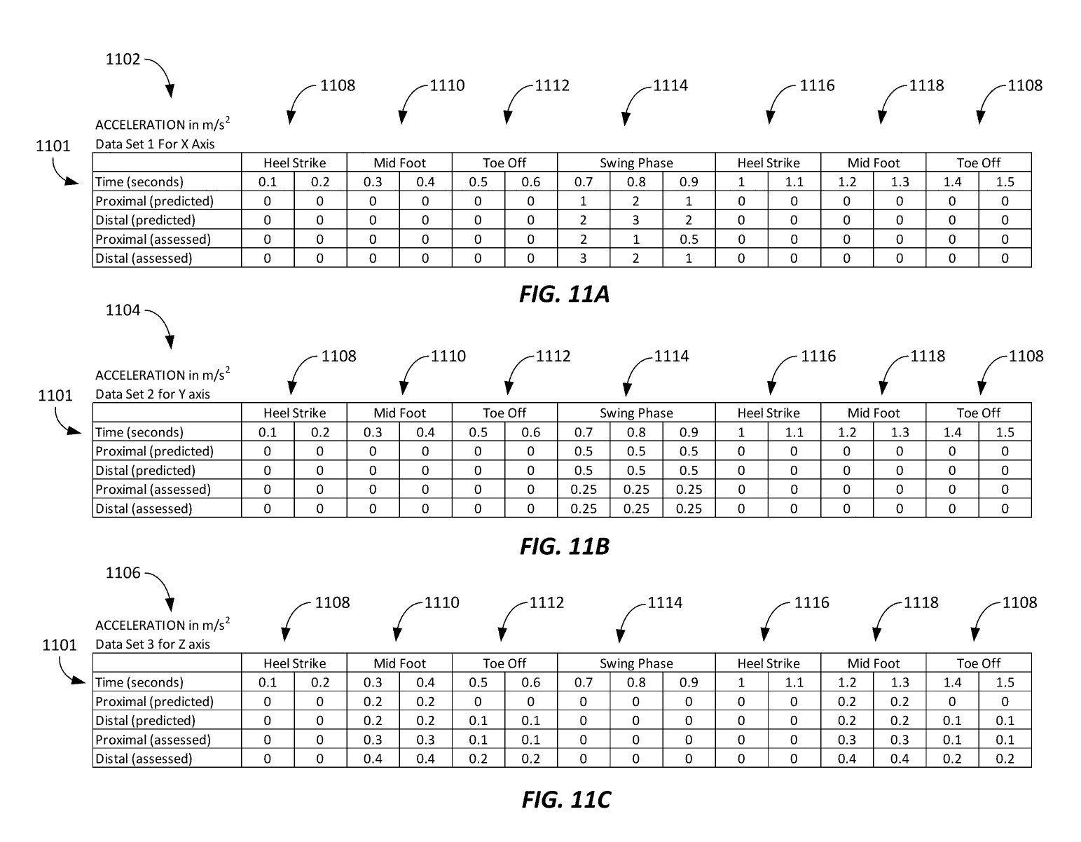

The current walk data (assessed) is compared to the baseline (predicted) data, for example, by charting or plotting the data. Referring to FIGS. 11A-C, table 1102 lists predicted and assessed acceleration data for X-axis movement at both the proximal and distal locations; table 1104 lists predicted and assessed acceleration data for Y-axis movement at both the proximal and distal locations; and table 1106 lists predicted and assessed acceleration data for Y-axis movement at both the proximal and distal locations. Each table has an identical and corresponding header row 1101 that lists the elapsed data collection time in seconds. Further, each table is divided into groups of columns in alignment with the individual gait motions, as determined from analysis of the three-dimensional movement data. Thus, column group 1108 reflects data for the heel strike motion at, in this example, 0.1 and 0.2 seconds; column group 1110 reflects data for the mid-foot motion at 0.3 and 0.4 seconds; column group 1112 reflects data for the toe-off motion at 0.5 and 0.6 seconds; column group 1114 reflects data for the swing phase at 0.7 through 0.9 seconds; column group 1116 starts the second revolution or stride and again reflects data for the heel strike motion at 1.0 and 1.1 seconds; column group 1118 reflects data for the mid-foot motion at 1.2 and 1.3 seconds; and column group 1120 reflects data for the toe-off motion at 1.4 and 1.5 seconds.

The three-dimensional data presented in chart 1100 can be evaluated by human or machine to determined where each portion of a gait begins and ends, for example.

Once the user's baseline data is received from a test walk or run, the user may want to extend the distance, for example, to go further. We can compare the live “assessed” data for the new walk/run with the baseline “predicted” data from the test walk/run, and from that comparison, we can evaluate changes in the knee.

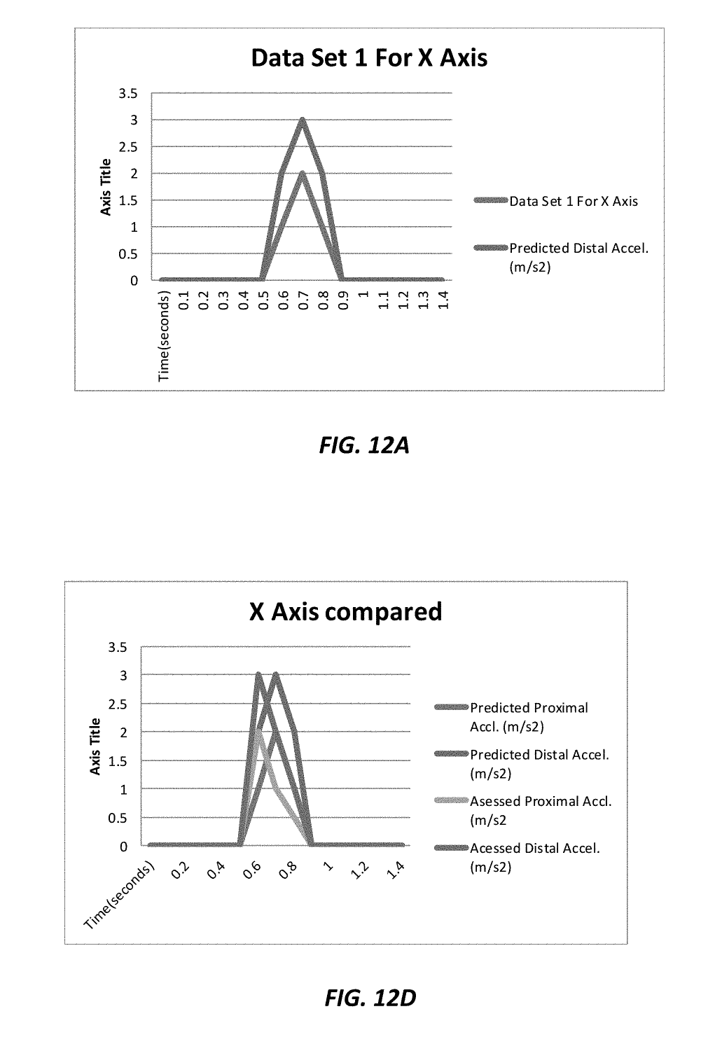

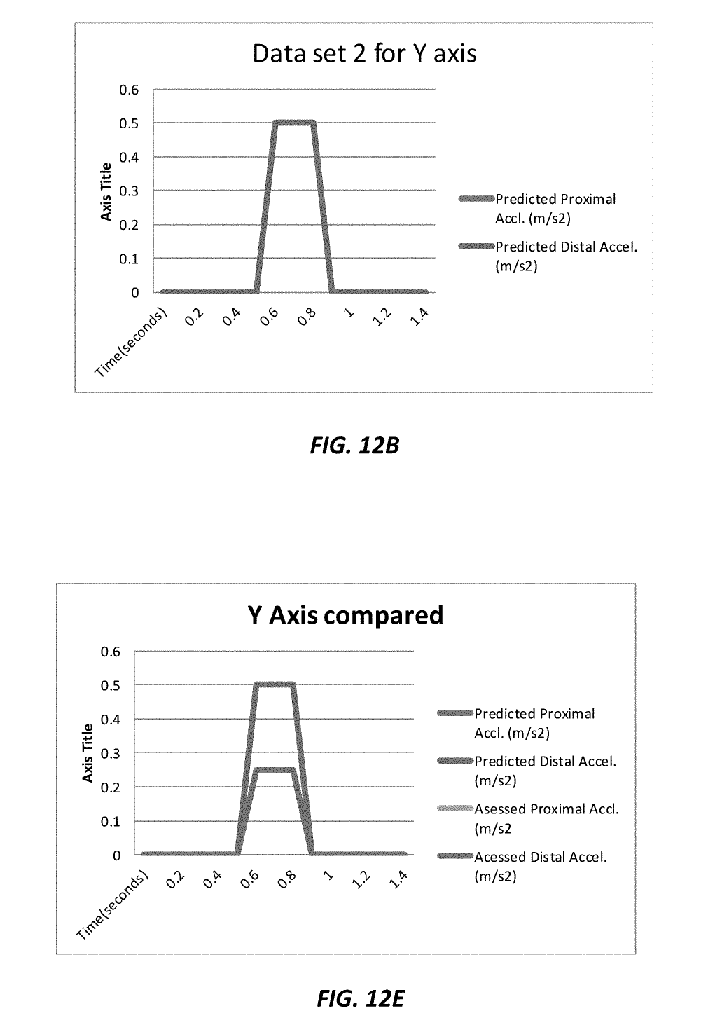

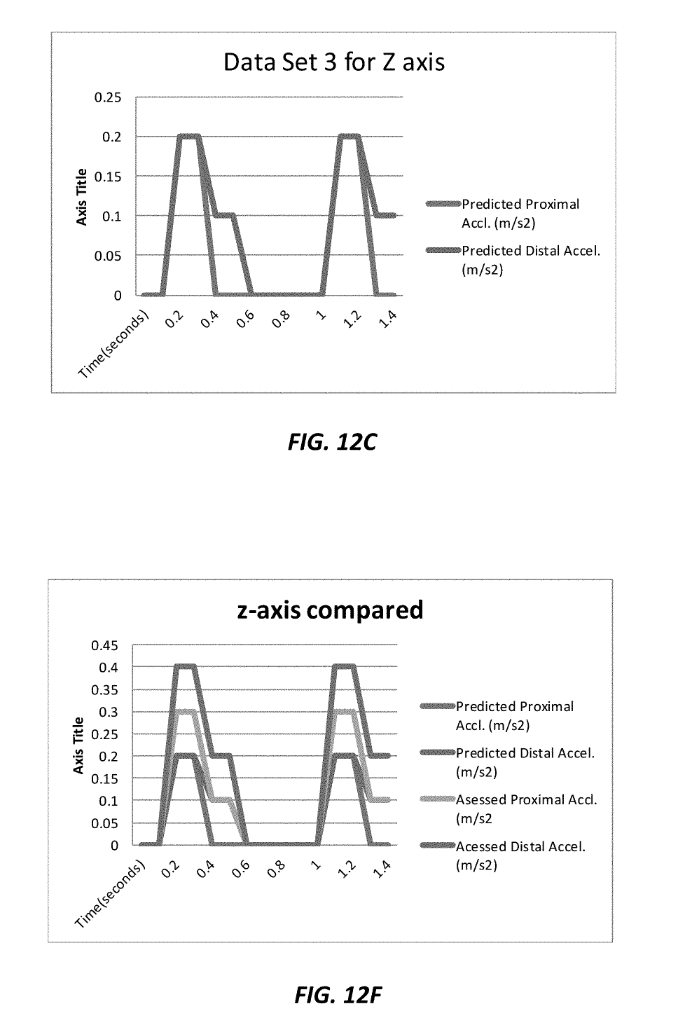

Referring now to FIGS. 12A-12E, the data from charts in FIG. 11 is plotted. The top set of three graphs indicates the predicted acceleration data for the X-axis (FIG. 12A), the predicted acceleration data for the Y-axis (FIG. 12B), and the predicted acceleration data for the Z-axis (FIG. 12C). The bottom set of three graphs include the plots for predicted data, but also include the assessed acceleration data for the X-axis (FIG. 12D), the assessed acceleration data for the Y-axis (FIG. 12E), and the assessed acceleration data for the Z-axis (FIG. 12F).

Thus, a visual comparison of the data can readily lead to some simple conclusions. For example, the predicted X-axis data (FIG. 12A) shows steady acceleration and deceleration at the toe-off and swing phases, which is normal and expected. The assessed X-axis data (FIG. 12S), however, indicates that the pace of the user is slowing. The slowing pace could be an indication that the user is not striding far enough, so one remedy would be tell the user to stop and stretch the hamstring. This is an example of the type of appropriate feedback that could be generated and provided to the user in real time based on the accelerometer data and a pre-programmed regimen. Of course, other indications and remedial exercises may be appropriate for different injuries, as determined by the user and the user's support group.

Looking now at the Y-axis data, the assessed plot (FIG. 12E) reveals that acceleration of the knee during the toe-off to swing phase transition is lower than the predicted plot (FIG. 12B), thus the user's knee is not raising as high. This may indicate hip flexor pain, as one example, the user could be told to stop and do a hip flexor stretch.

In the Z-direction, the assessed data (FIG. 12F) shows that acceleration has gone up compared to the predicted data (FIG. 12C), which indicates that the knee lacks some control due to muscle weakness or an increased environmental struggle. Another common example of this Z-axis movement is, in a squat exercise, the knee shakes, that is, it wiggles in and out when standing back up. The knee should stay straight ahead and the patella should track with the big toe.

Thus, there are many possible applications that would benefit from combining one or more accelerometers with one or more braces. Of course, the basic application described herein is simply to help avoid reinjury or the need for a joint replacement. To that end, the analysis can prescribe exercises, stretches, etc., that are targeted to the user's specific injury and rehabilitation. The availability of dynamic information directly from the relevant joint area helps the user and support group track, monitor and develop useful strategies that enhance and improve the rehabilitation process.

### 9. Conclusion

While one or more implementations have been described by way of example and in terms of the specific embodiments, it is to be understood that one or more implementations are not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

1. A knee brace, comprising:
a sleeve formed of flexible elastic material to conform to a human knee, the sleeve having a posterior section and an anterior section, the anterior section configured to conform to an anterior region of the human knee and cover a knee joint and formed with an increased volume of the flexible elastic material relative to the posterior section, the posterior section configured to conform to a posterior region of the human knee; and
at least one accelerometer affixed with the sleeve, wherein the accelerometer obtains and transmits data regarding movement of the human knee to a processor-based device.

2. The knee brace of claim 1, the anterior section further comprising:
a lateral section formed at an outside edge of the anterior section and configured to conform to a lateral region extending around an outside anterior portion of the knee joint;
wherein the accelerometer is affixed to the lateral section.

3. The knee brace of claim 2, further comprising:
a first accelerometer affixed near a top of the lateral section; and
a second accelerometer affixed near a bottom of the lateral section.

4. The knee brace of claim 1, the anterior section further comprising:
a medial section formed at an inside edge of the anterior section and configured to conform to a medial region extending around an inside anterior portion of the knee joint;
wherein the accelerometer is affixed to the medial section.

5. The knee brace of claim 4, further comprising:
a first accelerometer affixed near a top of the medial section; and
a second accelerometer affixed near a bottom of the medial section.

6. The knee brace of claim 3, the anterior section further comprising:
a medial section formed at an inside edge of the anterior section and configured to conform to a medial region extending around an inside anterior portion of the knee joint;
a third accelerometer affixed near a top of the medial section; and
a fourth accelerometer affixed near a bottom of the medial section.

7. The knee brace of claim 1, wherein the processor-based device is a smartphone.

8. The knee brace of claim 7, wherein the smartphone includes an additional accelerometer.

9. The knee brace of claim 7, wherein the smartphone includes an application for processing the movement data received from the accelerometer.

10. The knee brace of claim 9, wherein the accelerometer transmits the movement data via a Bluetooth communications protocol.

11. A knee brace system, comprising:
a first sleeve formed of flexible elastic material to conform to a human knee, the first sleeve including:
a first upper ring configured to be secured above the human knee;
a first lower ring configured to be secured below the human knee;
a first anterior panel affixed between the first upper ring and the first lower ring and configured to conform to an anterior region of the human knee,
a first posterior panel affixed between the first upper ring and the first lower ring and configured to conform to a posterior region of the human knee, wherein the first anterior panel is formed with an increased volume of the flexible elastic material relative to the first posterior panel;
a first lateral panel affixed between the first upper ring and the first lower ring and between the first anterior panel and the first posterior panel, the first lateral panel configured to conform to an outside portion of the human knee; and
a first pair of accelerometers affixed in the first lateral panel, wherein a first one of the first pair of accelerometers is affixed near the first upper ring and a second one of the first pair of accelerometers is affixed near the first lower ring.

12. The knee brace system of claim 11, further comprising:
a first medial panel affixed between the first upper ring and the first lower ring and between the first anterior panel and the first posterior panel, the first medial panel configured to conform to an inside portion of the human knee; and
a second pair of accelerometers affixed in the first medial panel, wherein a first one of the second pair of accelerometers is affixed near the first upper ring and a second one of the first pair of accelerometers is affixed near the first lower ring.

13. The knee brace system of claim 11, further comprising:
a second sleeve formed of flexible elastic material to conform to a human knee, the second sleeve including:
a second upper ring configured to be secured above the human knee;
a second lower ring configured to be secured below the human knee;
a second anterior panel affixed between the second upper ring and the second lower ring and configured to conform to an anterior region of the human knee,
a second posterior panel affixed between the second upper ring and the second lower ring and configured to conform to a posterior region of the human knee, wherein the second anterior panel is formed with an increased volume of the flexible elastic material relative to the second posterior panel;
a first medial panel affixed between the second upper ring and the second lower ring and between the second anterior panel and the second posterior panel, the first medial panel configured to conform to an inside portion of the human knee; and
a second pair of accelerometers affixed in the first medial panel, wherein a first one of the second pair of accelerometers is affixed near the second upper ring and a second one of the second pair of accelerometers is affixed near the second lower ring.

14. The knee brace system of claim 11, wherein the accelerometers obtain and transmit data regarding movement of the human knee to a processor-based device.

15. The knee brace system of claim 11, wherein the processor-based device is a smartphone.

16. A knee brace system, comprising:
a first sleeve formed of flexible elastic material to conform to a human knee, the first sleeve including:
a first anterior panel configured to conform to an anterior region of the human knee,
a first posterior panel configured to conform to a posterior region of the human knee, wherein the first anterior panel is formed with an increased volume of the flexible elastic material relative to the first posterior panel;
a first lateral panel configured to conform to an outside portion of the human knee; and
a first pair of accelerometers affixed in the first lateral panel, wherein a first one of the first pair of accelerometers is affixed in an upper portion of the first lateral panel and a second one of the first pair of accelerometers is affixed in a lower portion of the first lateral panel; and
a second sleeve formed of flexible elastic material to conform to a human knee, the second sleeve including:
a second anterior panel configured to conform to an anterior region of the human knee,
a second posterior panel configured to conform to a posterior region of the human knee, wherein the second anterior panel is formed with an increased volume of the flexible elastic material relative to the second posterior panel;
a first medial panel configured to conform to an inside portion of the human knee; and
a second pair of accelerometers affixed in the first medial panel, wherein a first one of the second pair of accelerometers is affixed in an upper portion of the first medial panel and a second one of the second pair of accelerometers is affixed in a lower portion of the first medial panel;
wherein the accelerometers generate and transmit data regarding movement of the human knee to a processor-based device.

17. A method, comprising:
receiving, at a processor, acceleration data from at least a first accelerometer and a second accelerometer both affixed in a first knee brace being worn by a user during walking or running exercise, the first accelerometer is affixed in an above-the-knee portion of the first knee brace and the second accelerometer is affixed in a below-the-knee portion of the first knee brace;
creating a graphical plot, by the processor, of the acceleration data over time in three dimensions, the graphical plot illustrating relative movement of the above-the-knee portion and the below-the-knee portion of the first knee brace;
based on the graphical plot, evaluating a gait of the user during the exercise;
providing feedback to the user regarding the user's gait during exercise.

18. The method of claim 17, wherein the first accelerometer and second accelerometer are positioned in a lateral panel of the first knee brace.

19. The method of claim 18, further comprising:
receiving, at the processor, acceleration data from a third accelerometer and a fourth accelerometer, the third accelerometer is affixed in an above-the-knee portion of a medial panel of the first knee brace and the second accelerometer is affixed in a below-the-knee portion of a medial panel of the first knee brace.

20. The method of claim 17, further comprising:
receiving, at the processor, acceleration data from a fifth accelerometer positioned on a hip of the user, wherein the graphical plot illustrates relative movement of the user's hip and knee.
